(19) United States
(12) Patent Application Publication
(10) Pub. No.: US 2010/0240878 A1
(43) Pub. Date: Sep. 23, 2010

(54) Fluorescent saccharide conjugates

(76) Inventor: Donald L. Barbeau, Evanston, IL (US)

Correspondence Address: Donald L. Barbeau, 6 Williamsburg Circle, Evanston, IL, 60203, US

(21) Appl. No.: 12/383,178

(22) Filed: Mar. 20, 2009

(51) Int. Cl. C07H 15/26; C07H 1/00

(52) U.S. Cl. 536/17.4; 536/17.6; 536/123.1

(57) ABSTRACT

The present invention relates to improved fluorescently labeled monosaccharide and low molecular weight oligosaccharide conjugates for immunofluorescent staining, confocal microscopic imaging and flow cytometry/fluorescence activated cell sorting (FACS). These fluorophore conjugates target cells, components of cell surfaces and extracellular components; and are useful as probes for tumor targeting and cellular uptake.

### BACKGROUND OF THE INVENTION

Fluorescent probes are routinely used to identify and characterize populations of cells, cell surfaces and extracellular components through the use of immunofluorescent staining, fluorescence microscopy, confocal microscopic imaging and flow cytometry/fluorescence activated cell sorting (FACS). In some cases, fluorescent probes are directly conjugated to monoclonal antibodies that can specifically bind to cell surface proteins, lectins and carbohydrates. In other cases, fluorescent probes have been conjugated to high molecular weight polysaccharides for assessing tumor targeting and cellular uptake of corresponding drug-conjugated polysaccharides. These approaches each have limited capabilities including: overly high specificity for populations of cells, cell surfaces and extracellular components; lack of targeting to certain components of cell surfaces and extracellular components; and physicochemical properties that preclude their binding ability to target certain tumor cell surfaces.

It is an object of the present invention to provide fluorescently labeled conjugates as probes for tumor targeting and cellular uptake of low molecular weight oligosaccharide fluorophore conjugates.

It is another object of the present invention to provide fluorescently labeled conjugates for immunofluorescent staining, confocal microscopic imaging and flow cytometry/fluorescence activated cell sorting (FACS).

It is a further object of the present invention to provide fluorescently labeled monosaccharide and low molecular weight oligosaccharide conjugates having improved physicochemical properties that permit their binding ability to certain tumor cell surfaces and extracellular components.

### BRIEF SUMMARY OF THE INVENTION

The present invention relates to fluorescent probes comprising targetable conjugates of a fluorescent compound and glycosides having the formula

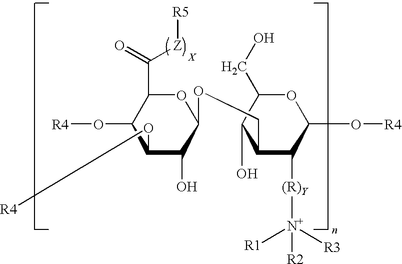

where R is an alkylene or heteroalkylene, Y=0 or 1, R₁, R₂ and R₃ are independently hydrogen, alkyl, aryl, aralkyl and cycloalkyl, R₄ is independently hydrogen, a monomeric glycoside, a dimeric glycoside and alkyl, X=0 or 1, Z is oxygen or a linker, n is from 1 to about 4 and R₅ is hydrogen or a fluorophore with the proviso that said compound contains at least one fluorophore.

### DETAILED DESCRIPTION OF THE INVENTION

In accordance with the present invention, the fluorescent conjugates comprise a fluorophore attached to monosaccharides or oligosaccharides having a molecular weight less than about 2,000 Daltons. In accordance with a preferred embodiment of the present invention, the fluorescent conjugates comprise a fluorophore covalently attached to water-soluble monosaccharides or to oligosaccharides having from 2 to about 8 glycoside residues. In a more preferred embodiment of the present invention, the fluorescent conjugates comprise a fluorophore covalently attached to water-soluble monosaccharides or to oligosaccharides having from 2 to about 6 glycoside residues.

Unless otherwise specifically identified or claimed for preferred embodiments, the following general definitions are used in accordance with the present invention.

In accordance with the present invention, the term fluorophore refers to a fluorescent chemical moiety which absorbs light at a given wavelength and emits light at a higher wavelength.

In accordance with the present invention, the term targetable refers to the recognition of a target by the conjugates. Targets include cell surfaces and extracellular components including, but not limited to tumor-specific antigens, negatively charged sialic acid residues on tumor cells, leukocytes and components of the extracellular matrix, and sialic acid on mucosal surfaces.

In accordance with the present invention, the term alkyl refers to a branched or straight chain acyclic alkyl group comprising one to about ten carbon atoms. In accordance with the present invention, the term lower alkyl refers to a branched or straight chain acyclic alkyl group comprising one to about six carbon atoms.

In accordance with the present invention, cycloalkyl refers to a saturated or unsaturated cyclic hydrocarbon comprising from about 3 to about 8 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo and nitro. In accordance with the present invention, cycloalkyl groups include cyclopropyl, cyclobutyl, cyclopentyl, cyclohexyl, cyclohexenyl, cycloheptyl and the like.

In accordance with the present invention, the term aryl refers to an unsubstituted or substituted monocyclic, bicyclic, carbocyclic or heterocyclic ring system comprising one or two aromatic rings. In accordance with the present invention, aryl groups include phenyl, pyridyl, napthyl, quinoyl, tetrahydronaphthyl, furanyl, indanyl, indenyl, indoyl, and the like.

In accordance with the present invention, the term arylalkyl refers to an aryl radical, attached to an alkyl radical in accordance with the present invention,

In accordance with the present invention tumor targeting and cellular uptake of the water-soluble low molecular weight oligosaccharide fluorescent conjugates can easily be determined by a number of conventional techniques described in the scientific literature including immunofluorescent staining, fluorescence microscopy, confocal microscopic imaging and flow cytometry/fluorescence activated cell sorting (FACS). Many of the classical fluorescent probes that have been successfully utilized in confocal microscopic imaging and flow cytometry/fluorescence activated cell sorting (FACS) include fluorescein isothiocyanate (FITC), rhodamine analogs, and Texas Red.

Fluorescein is a xanthene dye that has a high quantum yield, an absorption maximum at 495 nanometers and coincides quite well with the 488 nanometer (blue) spectral line produced by argon-ion and krypton-argon lasers, as well as the 436 of the mercury lamps and the 467 principal lines of the xenon arc-discharge lamps. Rhodamine dyes include, Rhodamine 123, Rhodamine B (tetraethylrhodamine), tetramethylrhodamine isothiocyanate (TRITC), NHS-Rhodamine, Rhodamine 116, Rhodamine 110, and Rhodamine 6G. TRITC is the base tetramethylrhodamine molecule functionalized with an isothiocyanate reactive group (—N═C═S) at one of two hydrogen atoms on the bottom ring of the structure. NHS-Rhodamine is activated with the N-hydroxy-succinimidyl-ester (NHS ester) functional group. NHS-ester derivative has high specificity toward primary amines, and results in a more stable linkage following labeling. Texas Red is a long-wavelength derivative of rhodamine that is modified with sulfonyl chloride for reaction to primary amines.

BODIPY fluorophores have spectral characteristics different than those of fluorescein, tetramethylrhodamine, Texas Red and longer-wavelength dyes. These fluorophores are useful as conjugates of proteins, nucleotides, oligonucleotides and dextrans, as well as to prepare fluorescent enzyme substrates, fatty acids, phospholipids, lipopolysaccharides, receptor ligands and polystyrene microspheres. BODIPY is an unsubstituted dipyrromethene referred to as 4,4-difluoro-4-bora-3a,4a-diaza-s-indacene. BODIPY-FL is comprised of dipyrromethene complexed with a disubstituted boron atom, typically a BF2 unit, and is called 4,4-difluoro-5,7-dimethyl-4-bora-3a,4a-diaza-/s/-indacene.

The use of the fluorophores in fluorescence-activated cell sorting (FACS) and immunohistochemical analysis is well-known and is clearly documented in the scientific and patent literature as evidenced by the following United States Patents which are hereby incorporated by reference: U.S. Pat. No. 7,505,618; U.S. Pat. No. 4,395,397; U.S. Pat. No. 4,629,687; U.S. Pat. No. 5,035,693; U.S. Pat. No. 4,284,897; U.S. Pat. No. 5,381,224; U.S. Pat. No. 5,646,41 1; U.S. Pat. No. 5,672,880; U.S. Pat. No. 5,7 19,391; U.S. Pat. No. 4,532,402; U.S. Pat. No. 5,690,846; U.S. Pat. No. 5,296,963; U.S. Pat. No. 5,932,872 and U.S. Pat. No. 7,462,696.

The preparation and use of the fluorophore probes in fluorescence-activated cell sorting (FACS), fluorescent immunohistochemical analysis and fluorophore conjugates targeted to tumor cells is well-known and is documented in the scientific literature including, Luo et al. Biomacromolecules 1:208-218 (2000); Pouyani et al. Bioconjugate Chemistry 5:370-372 (1994); Prestwich et al. Journal of Controlled Release 53 :93-103 (1998); Luo et al. Bioconjugate Chemistry 10: 755-753 (1999); Wortzel et al. EP 1991587 (WO 2007/102149) and Pouyani et al. Bioconjugate Chemistry 5:339-347 (1994).

In accordance with the present invention, disclosed are targetable fluorophore conjugates of having the formula

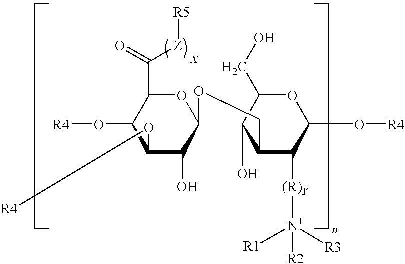

where R is an alkylene or heteroalkylene, Y=0 or 1, R₁, R₂ and R₃ are independently hydrogen, alkyl, aryl, aralkyl and cycloalkyl, R₄ is independently hydrogen, a monomeric glycoside, a dimeric glycoside or alkyl, X=0 or 1, Z is oxygen or a linker, n is from 1 to about 4 and R₅ is hydrogen or a fluorophore with the proviso that said compound contains at least one fluorophore.

In accordance with the present invention, R is an alkylene containing only carbon atoms or a heteroalkylene containing carbon, nitrogen and oxygen atoms. Examples of heteroalkylenes include —NHC(O)CH₂— and —NHC(O)CH₂CH₂—. In accordance with the present invention, R is an alkylene having from 1 to about 6 carbon atoms. In a preferred embodiment of the present invention, R is a lower alkylene having from 1 to about 3 carbon atoms. In accordance with a more preferred embodiment of the present invention, R is —CH₂ or —CH₂CH₂—. In accordance with the present invention, Y is either 0 or 1. In accordance with a preferred embodiment of the present invention, Y is 0.

In accordance with the present invention, R₁, R₂ and R₃ are independently hydrogen, alkyl, aryl and cycloalkyl. Preferred alkyls are those having from 1 to about 6 carbon atoms including but not limited to methyl, ethyl, propyl, butyl, isobutyl, pentyl and hexyl. More preferably the alkyls contain from 1 to about 3 carbon atoms. In accordance with the present invention, R₁, R₂ and R₃ are each methyl or ethyl. Preferred aryls are those including but not limited to phenyl and pyridinyl, while a preferred aralkyl is benzyl.

Preferred cycloalkyls are those having from about 3 to about 6 carbon atoms including but not limited to cyclopropyl, cyclobutyl, cyclopentyl, and cyclohexyl.

In accordance with the present invention, R₄ is independently hydrogen, a monomeric glycoside, a dimeric glycoside or alkyl. In accordance with one aspect of the present invention R₄ is an alkyl having from 1 to about 6 carbon atoms. In a preferred embodiment of the present invention, R₄ is independently hydrogen, methyl, ethyl or propyl. In accordance with the present invention, illustrative dimeric glycosides include, but are not limited to, compounds having the formula

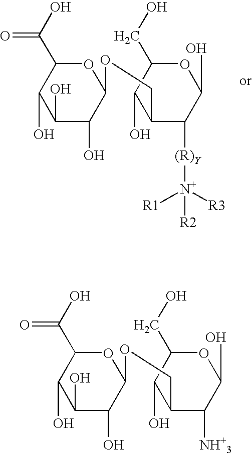

In accordance with this embodiment of the present invention, the oligosaccharides include multiple glycosides such that n is an integer from about 1 to about 4. In a preferred embodiment of the present invention, the conjugates include an oligosaccharide where n is an integer from 1 to about 3.

In accordance with the present invention, the linker can be any suitable chemical group that permits conjugation of the fluorescent compound with the carboxyl or hydroxyl group of the saccharides including, but not limited to, hydrazides, hydrazones, succinates, adipates, suberates, and adipic dihydrazide.

Illustrative compounds in accordance with another aspect of the present invention include, inter alia, fluorophore-oligosaccharide conjugates having the formula:

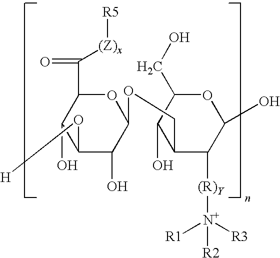

where n=1 to about 4;
fluorophore-oligosaccharide conjugates having the formula

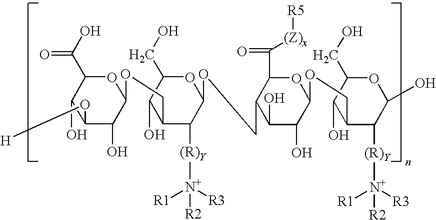

where n=1 or 2, and fluorophore-oligosaccharide conjugates having the formula

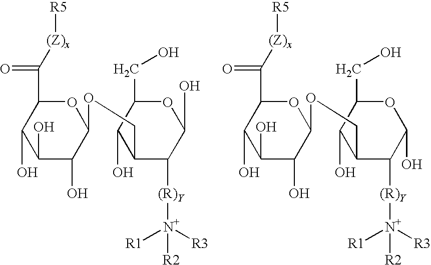

Illustrative compounds in accordance with another aspect of the present invention include, inter alia, fluorescein-oligosaccharide conjugates having the formula:

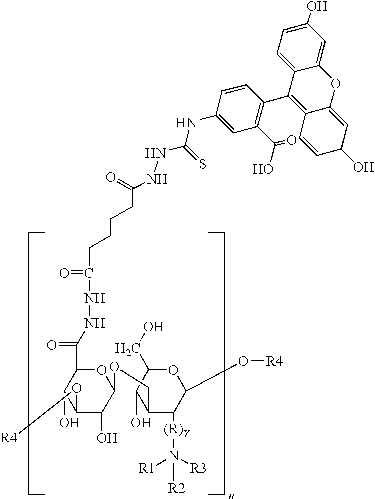

Illustrative compounds in accordance with another aspect of the present invention include, inter alia, Texas Red-oligosaccharide conjugates having the formula:

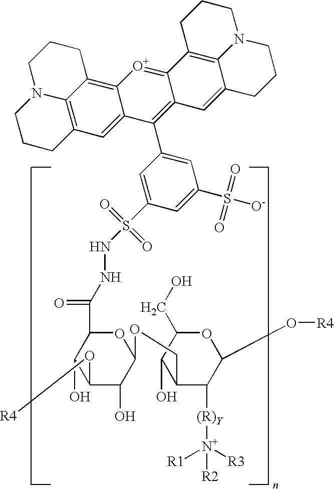

In another aspect of the present invention, disclosed are monosaccharide drug conjugates having the formula

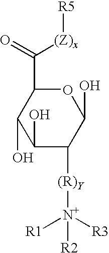

where R is an alkylene or heteroalkylene, Y=0 or 1, R₁, R₂ and R₃ are independently hydrogen, alkyl, aryl, aralkyl and cycloalkyl, X=0 or 1, Z is oxygen or a linker and R₅ is a fluorophore. In a preferred embodiment of the present invention, the fluorophore is conjugated to 2-amino-D-glucuronic acid (CAS No. 50767-834).

The present invention has been described in detail using specific examples to illustrate the preferred embodiments of the invention; however, it will be obvious to those skilled in the art that various modifications thereto can be made without departing from the spirit and scope thereof.

I claim:

1. A compound having the formula 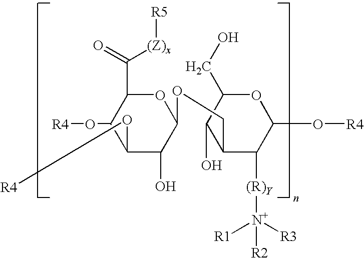
where R is an alkylene or heteroalkylene, Y=0 or 1, R₁, R₂ and R₃ are independently hydrogen, alkyl, aryl, aralkyl and cycloalkyl, R₄ is independently hydrogen, a monomeric glycoside, a dimeric glycoside or alkyl, X=0 or 1, Z is oxygen or a linker, n is from 1 to about 4 and R₅ is hydrogen or a fluorophore with the proviso that said compound contains at least one fluorophore.

2. The compound of claim 1 having the formula

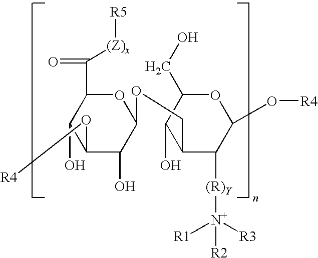

3. The compound of claim 1 wherein said fluorophore is fluorescein isothiocyanate, a rhodamine dye or Texas Red.

4. The compound of claim 2 wherein said fluorophore is fluorescein isothiocyanate, a rhodamine dye or Texas Red.

5. The compound of claim 2 wherein R₁, R₂ and R₃ are hydrogen.

6. The compound of claim 2 wherein R₁, R₂ and R₃ are independently alkyl, aryl, aralkyl and cycloalkyl.

7. The compound of claim 2 having the formula

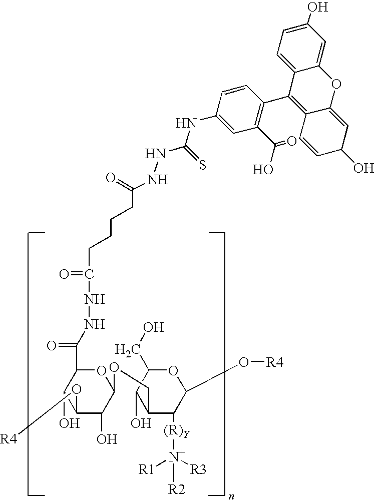

8. The compound of claim 2 having the formula

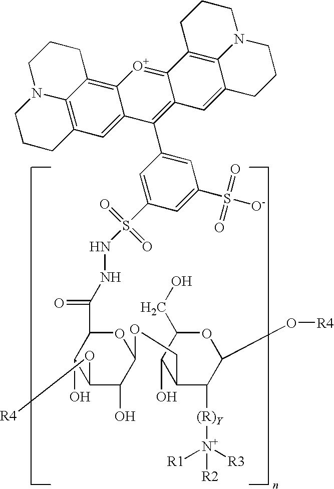

9. The compound of claim 1 having the formula

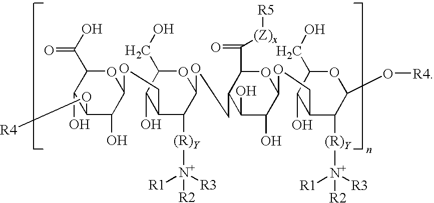

10. A compound having the formula 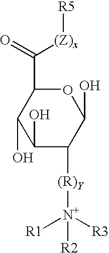
where R is an alkylene or heteroalkylene, Y=0 or 1, R₁, R₂ and R₃ are independently hydrogen, alkyl, aryl, aralkyl and cycloalkyl, X=0 or 1, Z is oxygen or a linker and R₅ is a fluorophore.

11. The compound of claim 10 wherein said fluorophore is fluorescein isothiocyanate, a rhodamine dye or Texas Red.

12. The compound of claim 10 wherein said fluorophore is fluorescein isothiocyanate, a rhodamine dye or Texas Red.

13. The compound of claim 10 wherein R₁, R₂ and R₃ are hydrogen.

14. The compound of claim 10 wherein R₁, R₂ and R₃ are independently alkyl, aryl, aralkyl and cycloalkyl.

15. A compound having the formula 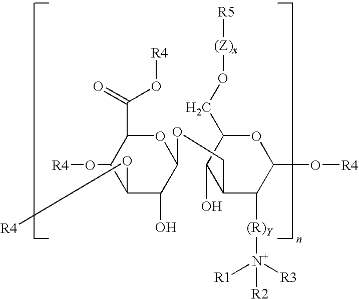
where R is an alkylene or heteroalkylene, Y=0 or 1, R₁, R₂ and R₃ are independently hydrogen, alkyl, aryl, aralkyl and cycloalkyl, R₄ is independently hydrogen, a monomeric glycoside, a dimeric glycoside or alkyl, X=0 or 1, Z is oxygen or a linker, n is from 1 to about 4 and R₅ is hydrogen or a fluorophore with the proviso that said compound contains at least one fluorophore.

16. The compound of claim 15 having the formula

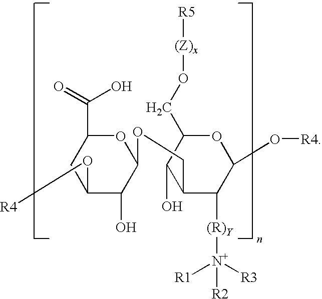

17. The compound of claim 15 wherein said fluorophore is fluorescein isothiocyanate, a rhodamine dye or Texas Red.

18. The compound of claim 15 wherein said fluorophore is fluorescein isothiocyanate, a rhodamine dye or Texas Red.

19. The compound of claim 16 wherein R₁, R₂ and R₃ are hydrogen.

20. The compound of claim 16 wherein R₁, R₂ and R₃ are independently alkyl, aryl, aralkyl and cycloalkyl.
